(19) United States
(12) Patent Application Publication
(10) Pub. No.: US 2014/0303482 A1
(43) Pub. Date: Oct. 9, 2014

(54) MAGNETIC RESONANCE IMAGING METHOD FOR IMAGING COMPONENTS WITH SHORT TRANSVERSE RELAXATION TIMES (T2) IN A HUMAN OR AN ANIMAL HEART

(71) Applicants: Francesco SANTINI, Basel (CH); Xeni DELIGIANNI, BASEL (CH); Oliver BIERI, Liestal (CH)

(72) Inventors: Francesco SANTINI, Basel (CH); Xeni DELIGIANNI, BASEL (CH); Oliver BIERI, Liestal (CH)

(21) Appl. No.: 13/856,121

(22) Filed: Apr. 3, 2013

(51) Int. Cl. A61B 5/055; G01R 33/561

(52) U.S. Cl. CPC A61B 5/055; G01R 33/561
USPC 600/411

(57) ABSTRACT

A magnetic resonance imaging (MRI) method for imaging components with short transverse relaxation times (T₂) is provided, in which a human or an animal heart is subjected to a segmented spoiled gradient echo (SPGR) sequence. Each segment of this SPGR sequence comprises a plurality of basic sequence elements in each of which a radiofrequency (RF) pulse and a frequency encoding gradient moment kx are applied, in order to generate an MRI signal at an echo time TE₁. The RF pulses and the frequency encoding gradient moments kx are applied such, that in different basic sequence elements the MRI signal is generated at varying echo times TE₁, in order to reduce the effective echo time in the center of k-space. The segments of the SPGR sequence are synchronized with at least one measured cycle indicator reflecting the timing of the cardiac cycles. The MRI signals generated by the SPGR sequence are used for reconstructing at least one first cardiac image.

### TECHNICAL FIELD

The present invention concerns a magnetic resonance imaging (MRI) method for imaging components with short transverse relaxation times (T₂) in a human or an animal heart. The method is particularly suited for the detection of myocardial fibrosis. The present invention also concerns a computer program comprising executable instructions for carrying out such an MRI method as well as an MRI system configured for carrying out such an MRI method.

### PRIOR ART

Imaging of components with short transverse relaxation times (T₂) by means of magnetic resonance imaging (MRI) methods represents a challenging task due to the very fast signal decay of these components after excitation of their nuclear magnetic spins. Imaging and detection of components with short transverse relaxation times (T₂) by means of MRI techniques, however, is desirable, because many of these tissue components that are important for clinical assessment appear black in conventional MRI due to their highly oriented structures. Such tissue components include for example the menisci, tendons and ligaments. As a consequence, the obtained magnetic resonance (MR) images are prone to misinterpretations.

An MRI method particularly developed for imaging components with short transverse relaxation times is the ultrashort echo time (UTE) imaging technique (Robson M D, Gatehouse P D, Bydder M, Bydder G M. Magnetic resonance: an introduction to ultrashort TE (UTE) imaging. J Comput Assist Tomogr 2003; 27: 825-846 and Bergin C J, Pauly J M, Macovski A. Lung parenchyma: projection reconstruction M R imaging. Radiology 1991; 179:777-781). One drawback of UTE sequences, however, is the radial acquisition scheme which not only requires an extended sampling time, but is also more prone to artefacts due to gradient system imperfections, as compared to Fourier-encoded, i.e. Cartesian sampling techniques. Moreover, the patient frequently needs to be repositioned with respect to the isocenter of the main magnetic field B₀ to circumvent issues arising from B₀ field shimming. Due to these reasons, UTE methods are hardly used in clinical routine.

A method which allows imaging of musculoskeletal fibrous tissue components and thus of components with very short T₂ relaxation times has been disclosed by Deligianni X, Bar P, Scheffler K, Trattnig S and Bieri O in 2012 in Magnetic Resonance in Medicine: High-resolution Fourier-encoded sub-millisecond echo time musculoskeletal imaging at 3 Tesla and 7 Tesla. With this method, the echo time of a Fourier-encoded spoiled gradient echo (SPGR) sequence is minimized, in order to image fibrous tissue components. The minimization of the echo time is achieved in particular by using a variable echo time (vTE) depending on the phase and slice encoding gradient moments applied after each radiofrequency (RF) pulse of the SPGR sequence.

A tissue component having a short transverse relaxation time (T₂) is collagen. An increased presence of collagen is a characteristic of cardiac fibrosis. The detection of myocardial fibrosis in the human or the animal body plays an important role in the clinical diagnosis of various heart diseases and in particular in patients suffering from myocardial infarction. The UTE- and vTE-techniques disclosed in the prior art documents mentioned above are not suited for imaging the heart, the first due to the resulting motion artefacts and the second because it is specifically adapted to MSK imaging.

An MRI method which allows the detection of myocardial fibrosis is late gadolinium enhancement (LGE) in combination with the application of an inversion recovery sequence (Pennell D J, Sechtem U P, Higgins C B, et al. Clinical indications for cardiovascular magnetic resonance (CMR): Consensus Panel report. Eur. Heart J. 2004/11//2004; 25(21):1940-1965, Kim R J, Chen E L, Lima J A, Judd R M. Myocardial Gd-DTPA kinetics determine MRI contrast enhancement and reflect the extent and severity of myocardial injury after acute reperfused infarction. Circulation 1996; 94:3318-26.). The signal of normal myocardium is suppressed by the inversion recovery sequence, such that the detection of the (enhanced) areas of the injured myocardium is improved. However, late gadolinium enhancement requires the administration of a contrast agent and does not allow to discriminate between (sub)acute and chronic myocardial injuries. Moreover, standard inversion recovery sequences suppress the signal of the normal myocardium, but not of the blood pool inside the ventricles. Therefore, delineation of injured myocardium with regard to the blood pool in these images represents a difficult task, even for an experienced radiologist.

### SUMMARY OF THE INVENTION

It is an object of the present invention to provide a magnetic resonance imaging (MRI) method for imaging components with short transverse relaxation times (T₂) in a human or an animal heart without the necessity to administer a contrast agent.

In the following, a short transverse relaxation time (T₂) component in a human or an animal heart is considered to be represented by a T₂ value of less than 10 ms, particularly of less than 5 ms and even more particularly of less than 2 ms.

The present invention provides a magnetic resonance imaging (MRI) method for imaging components with short transverse relaxation times (T₂) in a human or an animal heart, in particular for imaging myocardial fibrosis, comprising at least the following steps:
subjecting the human or the animal heart to a spoiled gradient echo (SPGR) sequence, the SPGR sequence being segmented into segments, wherein each segment comprises a plurality of basic sequence elements in each of which a radiofrequency (RF) pulse and a frequency encoding gradient moment kx are applied, in order to generate a magnetic resonance (MR) signal at an echo time TE₁ after the RF pulse;
applying the RF pulses and the frequency encoding gradient moments kx such, that in different basic sequence elements the MR signal is generated at varying echo times TE₁, in order to reduce the effective echo time in the center of k-space;
measuring at least one cardiac cycle indicator which reflects the timing of the cardiac cycles of the human or of the animal heart;
synchronizing the segments of the SPGR sequence with the measured cardiac cycle indicator;
acquiring the MR signals generated at the varying echo times TE₁; and
reconstructing at least one first cardiac image based on the acquired MR signals.

By applying a segmented SPGR sequence with variable echo times TE₁ and synchronizing the segments of this sequence with the measured cardiac cycle indicator, an effective echo time can be achieved, which allows imaging components with short transverse relaxation times (T₂) even in a beating human or animal heart without the need of administering a contrast agent. Due to the synchronization of the SPGR sequence with the cardiac cycle indicator (ECG-gating), motion artefacts can be avoided to a large extent. Thus, imaging of components with short T₂ values, such as of collagen in the heart and, as a consequence, the detection of myocardial fibrosis becomes possible.

The heart is usually in a living human or animal body and, therefore, moving repetitively and essentially regularly or, due to certain diseases, moving irregularly.

The measuring principle of spoiled gradient echo (SPGR) sequences, which are also known by the terms FLASH or T₁-FFE depending on the manufacturer of the MRI system, is usually characterized by the application of a series of consecutive radiofrequency (RF) pulses, with a repetition time interval TR between each of two consecutive RF pulses that is shorter than or in the same order of magnitude of the transverse relaxation time T₂ of the sample to be measured. Each of these RF pulses belongs to one basic sequence element. In order to suppress signals from previous RF excitations, a dephasing gradient moment is usually applied in frequency encoding (also called readout), phase encoding and/or slice selection direction prior to each RF pulse. RF spoiling can additionally be applied, in order to suppress MR signals from previous excitations. Phase encoding gradient moments ky which are normally applied in the basic sequence elements of the SPGR sequence for enabling a complete spatial reconstruction of the cardiac image, are usually rewound prior to the next RF excitation in the subsequent basic sequence element.

K-space is a term widely used in MRI and well known to the person skilled in the art and defines the spatial frequency domain of the measured image data in contrast to image space which is related with k-space by means of an (inverse) Fourier transformation. A reduction of the overall effective echo time in the center of k-space means that MR signals contributing to low spatial frequencies of the reconstructed first cardiac image are acquired at a reduced echo time TE₁ as compared to MR signals contributing to high spatial frequencies of the reconstructed first cardiac image. Thus, the generation and acquisition of MR signals at varying echo times TE₁ leads to a reduced overall mean echo time of the MR signals of all basic sequence elements as compared to a conventional acquisition with a constant echo time.

In order to avoid image artefacts due to imperfections of the gradient system, preferably Cartesian sampling is applied. Image acquisition can either be two-dimensional or three dimensional, i.e. acquisition of volumetric image data. Also possible is the acquisition of multi-slice two-dimensional images.

The segmented SPGR sequence and, as a consequence, the segmented approach for image data acquisition allows the acquisition of only a fraction of the total number of k-space lines during one heartbeat.

The cardiac cycle indicator is preferably determined based on a measurement of the electrocardiogram (ECG), usually by means of electrodes placed on the chest of the subject whose heart is to be examined. Preferably, the peaks of the R-waves of the ECG are detected and used as the cardiac cycle indicator. However, alternative methods for measuring a cardiac cycle indicator exist, such as for example pulse oximetry.

In order to avoid artefacts due to the motion of the diaphragm, the acquisition of the MR signals is preferably carried out during one or several breath-holds. Artefacts due to a misalignment of the diaphragm position in different breath-holds can be avoided, if the entire image acquisition is carried out during one single breath-hold only. This can particularly be achieved if the entire image acquisition is carried out in less than 25 heartbeats, more preferably less than 20 heartbeats and most preferably less than 15 heartbeats.

In order to acquire sufficient MR signals within a minimum number of breath-holds for enabling the reconstruction of a cardiac image with satisfying spatial resolution, the repetition time interval TR between two consecutive RF pulses of one segment is preferably shorter than 50 ms, more preferably shorter than 30 ms and most preferably shorter than 20 ms.

The flip angles of the RF pulses applied in the SPGR sequence are preferably in the range of 10° to 30°, more preferably in the range of 10° to 25°, and most preferably about 15°. The signal-to-noise ratio of the obtained cardiac image is maximized with these flip angles.

The MR signals are preferably phase encoded and/or slice encoded by means of corresponding phase encoding gradients Gy (generating a phase-encoding moment ky) and/or slice selection/partition encoding gradients Gz (generating a partition-encoding moment kz in case of three-dimensional acquisitions, or appropriately rewinded in case of multi-slice or two-dimensional acquisition) applied in the basic sequence elements of the SPGR sequence. The phase encoding gradient moments ky and/or slice slice selection or partition encoding gradient moments kz allow the encoding of the spatial signal location along directions perpendicular to the frequency encoding direction. The echo time TE₁ in each basic sequence element is advantageously dependent on the duration of the phase encoding gradient Gy or on the duration of the slice selection/partition encoding gradient Gz applied in the same basic sequence element. Preferably, in order to achieve a short echo time TE_{L} in each basic sequence element the gradient amplitudes and more preferably also the gradient slew rates are maximized within the given restrictions of the available MRI-system and/or within the constraints prescribed with respect to peripheral nerve stimulation, in particularly as the phase encoding gradient, the slice encoding gradient and/or the frequency encoding gradient are concerned. In each basic cardiac element the frequency encoding gradient Gx is preferably applied as soon as possible after the RF pulse. Thus, the echo time TE₁ of each basic sequence element preferably is directly dependent on the duration of the phase encoding gradient Gy and/or the slice encoding gradient Gz applied in the same basic sequence element. Thereby, the effective echo time in the center of k-space can be minimized.

Preferably, magnitudes of the gradient moments applied in phase encoding and/or in slice encoding direction are more than 32 mT/m, more preferable even more than 38 mT/m. The slew rates of the gradient moments are preferably more than 160 mT/m per ms.

In order to obtain a cardiac image reflecting signals from as many components with short T₂ values as possible, the effective echo time in the center of k-space is preferably shorter than 2 ms, more preferably shorter than 1.5 ms and most preferably even shorter than 1 ms.

Preferably, the echo time TE₁ is constant for ky<kₘᵢₙ or for kz<kₘᵢₙ, and the echo time TE₁ is a linear function of ky for ky≧kₘᵢₙ or a linear function of kz or for kz≧kₘᵢₙ, wherein kₘᵢₙ represents a gradient moment in the range between 0 and the maximum phase encoding or slice encoding gradient moment kₘₐₓ applied in the SPGR sequence. Thus, a particularly short and constant echo time TE₁ is achieved for low phase encoding and/or slice encoding moments, which results in a corresponding cardiac image, in which short T₂ components are particularly well discernible and analysable in the corresponding lower spectral spatial frequencies of the image. Longer echo times TE₁ are obtained for high phase encoding and/or slice encoding moments.

In a preferred embodiment, an additional MR signal is generated in each basic sequence element at an echo time TE₂ after the RF pulse, and the MR signals generated at the echo time TE₂ are used for reconstructing at least one second cardiac image. The echo time TE₂ is preferably constant over all basic sequence element and is advantageously in the range of 2 ms and 10 ms and more advantageously in the range of 3 ms and 7 ms. The MR signals arising at TE₂ and being used for reconstructing the second cardiac image are preferably generated in the form of a gradient echo with the frequency encoding gradient holding the same polarity as the one used to generate the signal at echo time TE₁.

The second cardiac image can be used to differentiate between components with short T₂ values and components with long T₂ values in the first cardiac image. To this end, the difference of the signal intensities of the first cardiac image and of the second cardiac image is preferably calculated. The obtained difference image then only reflects components with short T₂ values. The bandwidth per pixel is preferentially identical or at least nearly the same for the first and the second cardiac image.

Preferably, the MR signals generated at the echo time TE₁ and the MR signals generated at the echo time TE₂ are acquired such, that the phase of the nuclear spins of the fat protons and of the water protons is essentially the same during the acquisition of the corresponding MR signals. This allows an efficient suppression of the fat signal in the finally obtained difference image.

The SPGR sequence advantageously comprises fat presaturation pulses which are applied in at least a part, preferably a large part, and most preferably in each of the segments, in order to suppress the MRI signal of fat protons. Thereby, the fat signal, which often hampers image analysis in MRI due to its strong intensity as compared to the intensity of the tissue signal, can be reduced efficiently.

The MR signals are preferably acquired asymmetrically in frequency encoding direction. In other words, only a fraction of k-space data is acquired in frequency encoding direction, wherein the acquired fraction is asymmetric with respect to k-space origin. This technique is also known as Partial Fourier Imaging and is usually followed by partial Fourier reconstruction, in order to obtain a complete cardiac image. An asymmetric acquisition in frequency encoding direction allows achieving a shorter echo time TE₁ and leads to a reduction in imaging time.

Preferably, the SPGR sequence is synchronized with the measured cardiac cycle indicator such, that the MR signals used for the reconstruction of the at least one first cardiac image are generated at end diastole with respect to the cardiac cycle.

The SPGR sequence can comprise long-T₂ presaturation RF pulses which are applied in the form of long-T₂ suppression pulses, in order to suppress the MR signal of components with long transverse relaxation times (T₂).

In order to suppress the MR signal of fat, the RF pulses can be applied such, that they selectively excite water protons leaving fat protons essentially unexcited.

In order to reduce imaging time, the MR signals are generated and acquired such, that the reconstructed first cardiac image and/or the reconstructed second cardiac image reflects a rectangular field of view. In k-space, the corresponding cardiac image is undersampled in phase encoding and/or slice encoding direction.

Additionally, a magnetic resonance imaging (MRI) system is provided for imaging components with short transverse relaxation times (T₂) in a human or an animal heart according to a method as described, the MRI system at least comprising
a magnet for generating a main magnetic field at a location of a human or an animal heart to be imaged, in order to at least partly align nuclear spins of the heart;
an excitation module for applying radio frequency (RF) pulses to the heart, in order to excite the nuclear spins of the heart;
a gradient module for generating temporary magnetic gradient fields at a location of the heart;
an acquisition module for acquiring the magnetic resonance (MR) signals produced by excited nuclear spins of the sample;
a cardiac cycle measurement module for measuring at least one cardiac cycle indicator which reflects the timing of the cardiac cycles of the heart;
a control module configured for controlling the excitation module, the gradient module, the acquisition module and the cardiac cycle measurement module such, that the heart is subjected to a spoiled gradient echo (SPGR) sequence, the SPGR sequence being segmented into segments, wherein each segment is synchronized with the measured cardiac cycle indicator and comprises a plurality of basic sequence elements in each of which a radiofrequency (RF) pulse and a frequency encoding gradient moment kx are applied, in order to generate a magnetic resonance (MR) signal at an echo time TE₁ after the RF pulse, and wherein the RF pulses and the frequency encoding gradient moments kx are applied such, that in different basic sequence elements the MR signal is generated and acquired at varying echo times TE₁, in order to reduce the effective echo time in the center of k-space; and
a reconstruction module for reconstructing at least one image based on the acquired MR signals.

The control module or unit and the reconstruction module or unit can for example each be realized by hardware specifically dedicated for this purpose or by software being stored for example locally on a single device or distributed on multiple devices.

It is preferred, that the magnetic field B₀ generated by the main magnet is at least in the region of the sample essentially uniform. The magnitude of the magnetic field generated by the main magnet is preferably larger than 0.5 Tesla, more preferably larger than 1 Tesla, even more preferably larger than 2 Tesla and most preferably larger than 5 Tesla. With a stronger magnetic field, a better signal-to-noise ratio can be achieved, but imaging artefacts are also more pronounced.

Furthermore, a computer program, preferably stored on a storage device readable by a computer, is provided, for controlling a magnetic resonance imaging (MRI) system as described, in order to image components with short transverse relaxation times (T₂) in a human or an animal heart according to a method as described. The computer program comprises at least executable instructions to:
employ a spoiled gradient echo (SPGR) sequence on the MRI system, the SPGR sequence being segmented into segments, wherein each segment comprises a plurality of basic sequence elements in each of which a radiofrequency (RF) pulse and a frequency encoding gradient moment kx are applied, in order to generate a magnetic resonance (MR) signal at an echo time TE₁ after the RF pulse;
apply the RF pulses and the frequency encoding gradient moments kx such, that in different basic sequence elements the MR signal is generated at varying echo times TE₁, in order to reduce the effective echo time in the center of k-space of all basic sequence elements;
measure at least one cardiac cycle indicator which reflects the timing of the cardiac cycles of the human or of the animal heart;
synchronize the segments of the SPGR sequence with the measured cardiac cycle indicator;
acquire the MR signals generated at the varying echo times TE₁; and
reconstruct at least one first cardiac image based on the acquired MR signals.

Thus, the computer program carries out central parts of the method described above when executed in a processor of an MRI system or in a processor being connected with an MRI system. The computer program is usually realized as a computer program code element which comprises computer-implemented instructions to cause a processor to carry out a particular method. It can be provided in any suitable form, including source code or object code. In particular, it can be stored on a computer-readable medium or embodied in a data stream. The data stream may be accessible through a network such as the Internet.

### SHORT DESCRIPTION OF THE FIGURES

Preferred embodiments of the invention are described in the following with reference to the drawings, which only serve for illustration purposes, but have no limiting effects. In the drawings it is shown:

FIG. 1 shows a schematic illustration of an exemplary MRI system for carrying out the inventive MRI method;

FIG. 2 shows a schematic illustration of one sequence segment of the inventive MRI method in a R-R interval of the electrocardiogram (ECG);

FIG. 3A shows a basic sequence element (A) of the SPGR sequence of the inventive MRI method;

FIG. 3B shows a basic sequence element (B) of the SPGR sequence of the inventive MRI method;

FIG. 3C shows a basic sequence element (C) of the SPGR sequence of the inventive MRI method;

FIG. 4 shows the dependence of the echo time TE (TE₁) on the phase encoding gradient moment ky applied in the SPGR sequence of the inventive MRI method;

FIG. 5A shows the first cardiac image obtained at TE₁ by means of the inventive method in a healthy volunteer;

FIG. 5B shows the second cardiac image obtained at TE₂ by means of the inventive method in the same healthy volunteer as shown in FIG. 5A;

FIG. 5C shows the difference image obtained by subtracting the image intensities of the image shown in FIG. 5B from the image intensities of the image shown in FIG. 5A;

FIG. 6A shows a LGE image of a patient with suspected myocardial infarction, in a first short axis slice;

FIG. 6B shows a LGE image the same patient as in FIG. 6A, in a second short axis slice; and

FIG. 7 shows the difference image obtained by means of a method according to the invention, in a short axis slice of the heart of the same patient as in FIG. 6A.

### DESCRIPTION OF PREFERRED EMBODIMENTS

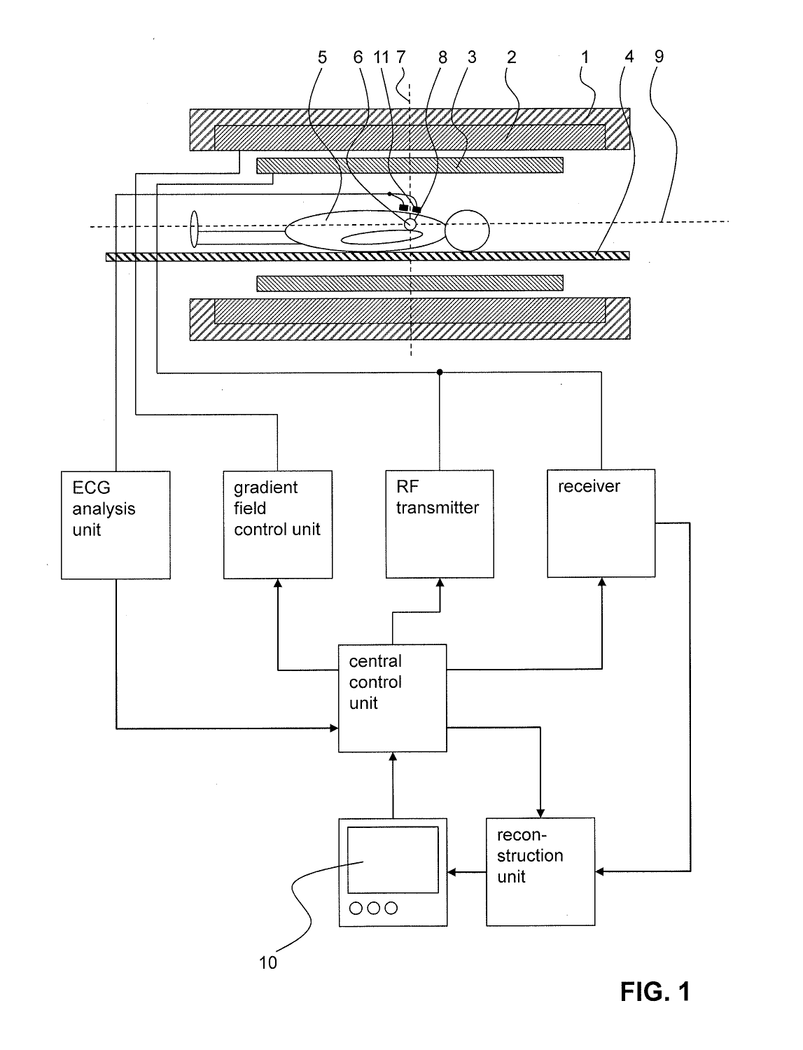

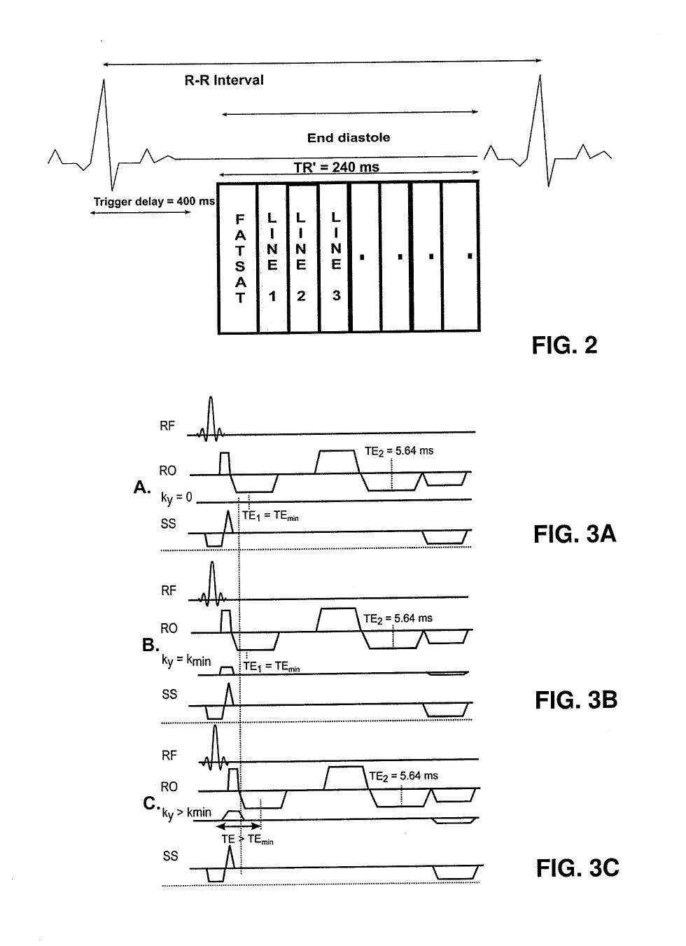

In FIG. 1, an exemplary MRI system is shown which serves to carry out the inventive method for imaging components with short transverse relaxation times (T₂) in a human or an animal heart. Preferred embodiments of the inventive method are schematically illustrated in FIGS. 2-4.

The MRI system comprises a main magnet 1 for producing a main magnetic field B₀. The main magnet 1 usually has the essential shape of a hollow cylinder with a horizontal bore. Inside the bore of the main magnet 1 a magnetic field is present, which is essentially uniform at least in the region of the isocenter 6 of the main magnet 1. The main magnet 1 serves to at least partly align the nuclear spins of a sample 5 arranged in the bore. Of course, the magnet 1 does not necessarily be cylinder-shaped, but could for example also be C-shaped.

A patient 5 is arranged in such a way on a moving table 4 in the bore of the main magnet 1, that the heart of the patient 5, of which components with short transverse relaxation times (T₂) are to be imaged, is arranged approximately in the region of the isocenter 6 of the magnet 1. In order to avoid motion artefacts due to diaphragm movement, the patient 5 is instructed to hold his breath during the entire image acquisition.

The main magnet 1 has a z-axis 9 which coincides with the central longitudinal axis defined by the cylindrical shape of the magnet 1. Together with a x-axis 7 and a y-axis 8, which each extend in mutually perpendicular directions with respect to the z-axis 9, the z-axis 9 defines a Cartesian coordinate system of the MRI system, having its origin at the isocenter of the magnet 1.

In order to produce a magnetic field which linearly varies in the direction of the x-axis 7, the y-axis 8 and/or the z-axis 9, the MRI-system comprises a gradient system 2 including several coils for producing these varying magnetic fields. A radiofrequency (RF) coil 3 and a RF transmitter connected with the RF coil 3 are provided for generating a transmit field B₁, in order to repetitively excite the nuclear spins of the patient 5 by means of RF pulses. The RF coil 3 is additionally connected with a receiver for the reception of the MR signals measured by the RF coil. Both the RF transmitter and the receiver are controlled by a central control unit.

ECG electrodes 11 placed on the chest of the patient 5 are adapted to send electric signals to an ECG analysis unit, in which preferably the timing of the R-waves of the ECG is detected. A corresponding cardiac cycle indicator reflecting the timing of the ECG R-wave is sent from the ECG analysis unit to the central control unit.

The receiver, which constitutes an acquisition module together with the RF coil 3, is connected with a reconstruction unit, in which the acquired MR signals are reconstructed into cardiac images. The cardiac images are sent from the reconstruction unit to a user interface 10, usually realized by a customary personal computer, in which the images are post processed.

For imaging components with short T₂ values in the heart of the patient 5, a segmented spoiled gradient echo (SPGR) sequence is initiated by an operator by means of a user interface 10, which sends the respective instructions to a central control unit of the MRI system (FIG. 1). The central control unit controls a gradient field control unit being connected with the gradient system 2 as well as the RF transmitter and the receiver both being connected with the RF coil 3. The gradient system 2 and the gradient field control unit together constitute a gradient module of the MRI system. The central control unit controls the gradient field control unit, the RF transmitter and the receiver based on the information received from the ECG analysis unit such, that an ECG-gated segmented SPGR imaging sequence is employed on the MRI system, which allows imaging components with short T₂ values.

The applied segmented SPGR sequence is illustrated in FIGS. 2 and 3. The R-wave of the ECG of the patient 5 is detected and used as a cardiac cycle indicator for the synchronization of the SPGR sequence with the heartbeat of the patient 5. During each heartbeat one segment of the SPGR sequence is applied with a trigger delay of 400 ms after the R-wave of the ECG, i.e. at end diastole. The duration TR′ of one entire segment of the SPGR sequence is here 240 ms.

As shown in FIG. 2, each segment of the applied SPGR sequence comprises a preparation part followed by a plurality of consecutive basic sequence elements (line 1, line 2, line 3 etc.). In the preparation part, a fat presaturation pulse in the form of a FAT SAT pulse is applied, in order to suppress the magnetic resonance (MR) signals of the fat protons in the entire segment. Further preparation steps are conceivable to be performed in each or in a part of the segments.

Following the fat presaturation pulse, a plurality of basic sequence elements is applied, in order to acquire multiple lines of the first cardiac image in k-space (line 1, line 2, line 3 etc.). Each line corresponds to one phase encoding step. Within one heartbeat (segment) only a fraction of the total number of lines used for reconstruction of the cardiac image can be acquired. After a certain number of heartbeats, however, sufficient lines are acquired, in order to obtain the cardiac image reflecting the components with short T2 values.

FIGS. 3A, 3B and 3C shows three basic sequence elements (A, B, C) of the SPGR sequence, in each of which one line of the first cardiac image is acquired in k-space at an echo time TE₁. The acquisition of lines A and B, and C as shown in FIGS. 3A, 3B and 3C, can be carried out in the same segment or in different segments of the SPGR sequence. During each RF pulse a gradient moment is applied in slice encoding (or slice selection, SS) direction, in order to only excite the nuclear spins of a certain slice, in particular of a short axis or long axis slice through the heart of patient 5. The RF pulse is followed by a gradient moment ky applied in phase encoding direction for encoding the spatial signal location along directions perpendicular to the frequency encoding direction. At the same time as the gradient moment ky, a gradient is applied on the z axis for the rephasing of the spins dephased by the slice selection gradient (rewinding gradient). The phase encoding gradient moment ky is increased in a plurality of incremental steps from ky=0 for the acquisition of line A to ky=kₘᵢₙ for acquiring line B. kₘᵢₙ is a gradient moment in the range between 0 and the maximum phase encoding gradient moment kₘₐₓ applied in the SPGR sequence. After the acquisition of line B, the phase encoding gradient moment is further increased to ky>kₘᵢₙ.

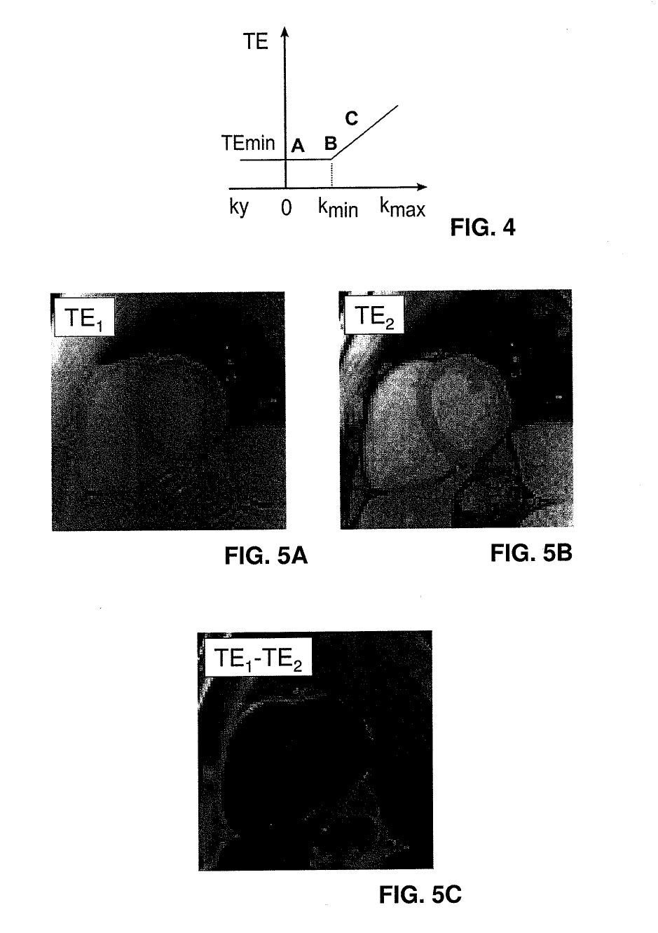

The duration of the phase encoding gradient is constant as long as ky≦kₘᵢₙ, because the variation of the gradient moments in the respective basic sequence elements is achieved by variations in the magnitude and/or the slew rate of the gradients and gradient time is bounded by the minimum achievable duration of the rewinding gradient. The gradient moment applied in frequency encoding (also referred to as readout, RO) direction is applied as soon as possible and immediately after the application of the RF pulse and of the phase encoding and slice encoding gradients. Since the duration of the RF pulse as well as the durations of the phase encoding and the slice encoding gradients is constant for ky≦kₘᵢₙ, the timing of the frequency encoding gradients is identical in all of the respective basic sequence elements. Hence, the MR signal is generated and acquired at a constant echo time TE₁=TEₘᵢₙ in these basic sequence elements with ky≦kₘᵢₙ (see FIG. 4, part A).

For the acquisition with ky=kₘᵢₙ, the magnitude and the slew rate of the phase encoding gradient are maximal due to limitations set by the MRI system or due to constraints with regard to peripheral nerve stimulation. Thus, for ky>kₘᵢₙ the variation of the phase encoding gradient moment is achieved by varying the duration of the respective gradients. As a result, the frequency encoding gradient applied in readout (RO) direction needs to be shifted in time, such that the corresponding MR signals are generated an acquired at an echo time TE₁>TEₘᵢₙ. As a consequence, the echo time TE₁ becomes a linear function dependent on the phase encoding gradient moment ky for ky>kₘᵢₙ (see FIG. 4, part C). For ky=kₘₐₓ, a maximal echo time TE₁=TEₘₐₓ results.

Due to the application of the SPGR sequence with varying echo times TE₁ as shown in FIGS. 3A, 3B, 3C and 4, the effective echo time in the center of k-space is significantly reduced, as compared to a SPGR sequence in which the MR signals are generated and acquired in all basic sequence elements at the same echo time TE₁=TEₘₐₓ.

Based on the MR signals acquired at TE₁, a first cardiac image is reconstructed, in which components with short T₂ values, in particular components with T₂>TE₁, are visible. For the reconstruction, the MR signals of all segments are combined, in order to yield a fully sampled image in the spectral spatial frequency domain, i.e. k-space, which is then transferred to image space by means of an (inverse) Fourier transformation. The person skilled in the art is well acquainted with performing such image reconstructions.

As can be seen from FIGS. 3A, B and C, a second MR signal is generated and acquired in each basic sequence element at an echo time TE₂=5.64 ms. The echo time TE₂ is constant throughout the entire SPGR sequence. The MR signal at echo time TE₂ is generated by means of additional gradient moments applied in frequency encoding direction, in order to induce a gradient echo at TE₂. Based on the MR signals acquired at TE₂, a second cardiac image is reconstructed, in which components with short T₂ values are visible to a much less extent as compared to the first cardiac image due to their natural T₂-decay. Components with long T₂ values, however, appear with nearly the same signal intensities in both the first and the second cardiac image.

In order to obtain an image free of components with long T₂ values, the signal intensities of the second cardiac image is subtracted from the corresponding signal intensities of the first cardiac image, which is usually carried out in the user interface 10 (FIG. 1). The difference image can then be used for the detection of myocardial fibrosis.

In a concrete measurement, a two-dimensional SPGR sequence with variable echo times was applied on a Siemens Magnetom Espree 1.5T MRI scanner within one breath hold in an ECG-gated mode and with data acquisition at end diastole. A rectangular field of view (FOV) of 342 mm×267 mm was defined for image acquisition yielding a voxel size of 1.8×1.8×5.5 mm (192×150 base image matrix, bandwidth per pixel=960 Hz). The duration of each of the 35 segments was 270 ms. The minimum TE₁ of the basic sequence elements for the acquisition of the first cardiac image was 0.79 ms, and the TE₂ for the acquisition of the second cardiac image was 5.64 ms. The RF pulse resulted in a flip angle of 15° and had a duration was 320 μs with a time-bandwidth product of 1.1. An asymmetric sampling of 29% was applied in frequency encoding direction and maximum gradient magnitudes and slew rates were applied. Three averages were acquired with identical sequence parameters, resulting in a total scan time of 15 s.

FIG. 5A shows the obtained first cardiac image based on the MR signals acquired at TE₁, and FIG. 5B shows the obtained second cardiac image based on the MR signals acquired at TE₂. FIG. 5C shows the differential image obtained by subtracting the signal intensities of the image shown in FIG. 5B from the image shown in FIG. 5A. Please note that for representation purposes, brightness and contrast levels have been adjusted independently in all images. The images shown in FIGS. 5A-5C were acquired in a healthy human volunteer. No components with short T₂ values are visible within the myocardium indicating that no myocardial fibrosis is present in the heart of this healthy volunteer.

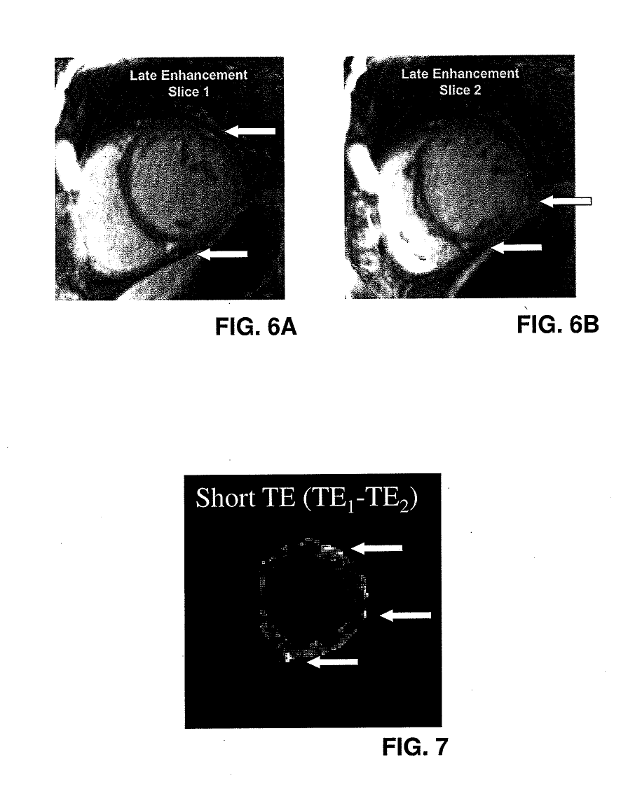

FIGS. 6A and 6B show short axis gadolinium late enhancement images for comparison purposes acquired on different levels of the heart of a patient with suspected myocardial infarction. Both images were acquired using a standard inversion recovery sequence after administration of a contrast agent. Late enhancement occurs within the myocardium at the positions indicated by the arrows, indicating the presence of myocardial fibrosis at these positions.

FIG. 7 shows the difference image obtained using the segmented SPGR sequence and after subtracting the second cardiac image (TE₂) from the first cardiac image (TE₁). Short T₂ components are visible within the myocardium at the positions indicated by the arrows, which is in good agreement with the positions of myocardial fibrosis as detected in the late enhancement images shown in FIGS. 6A and 6B.

### REFERENCE NUMERALS

| | |
|---|---|
| 1 | Main magnet |
| 2 | Gradient system |
| 3 | RF coil |
| 4 | Moving table |
| 5 | Patient |
| 6 | Isocenter |
| 7 | X-axis |
| 8 | Y-axis |
| 9 | Z-axis |
| 10 | User interface |
| 11 | ECG electrodes |

1. A magnetic resonance imaging (MRI) method for imaging components with short transverse relaxation times (T₂) in a human or an animal heart, comprising at least the following steps:
subjecting the human or the animal heart to a spoiled gradient echo (SPGR) sequence, the SPGR sequence being segmented into segments, wherein each segment comprises a plurality of basic sequence elements in each of which a radiofrequency (RF) pulse and a frequency encoding gradient moment kx are applied, in order to generate a magnetic resonance (MR) signal at an echo time TE₁ after the RF pulse;
applying the RF pulses and the frequency encoding gradient moments kx such, that in different basic sequence elements the MR signal is generated at varying echo times TE₁, in order to reduce the effective echo time in the center of k-space;
measuring at least one cardiac cycle indicator which reflects the timing of the cardiac cycles of the human or of the animal heart;
synchronizing the segments of the SPGR sequence with the measured cardiac cycle indicator;
acquiring the MR signals generated at the varying echo times TE₁; and
reconstructing at least one first cardiac image based on the acquired MR signals.

2. The method as claimed in claim 1, wherein the MR signals are phase encoded and/or slice encoded by means of corresponding phase encoding gradients Gy and/or slice encoding gradients Gz, and wherein the echo time TE₁ in each basic sequence element is dependent on the duration of the phase encoding gradient Gy or on the duration of the slice encoding gradient Gz applied in the same basic sequence element.

3. The method as claimed in claim 2, wherein the echo time TE₁ is constant for ky<kₘᵢₙ or for kz<kₘᵢₙ, and wherein the echo time TE₁ is a linear function of ky for ky≧kₘᵢₙ or a linear function of kz or for kz≧kₘᵢₙ, kₘᵢₙ representing a gradient moment in the range between 0 and the maximum phase encoding or slice encoding gradient moment kₘₐₓ applied in the SPGR sequence.

4. The method as claimed in claim 1, wherein an additional MR signal is generated in each basic sequence element at an echo time TE₂ after the RF pulse, and wherein the MR signals generated at the echo time TE₂ are used for reconstructing at least one second cardiac image.

5. The method as claimed in claim 4, wherein the difference of the signal intensities of the first cardiac image and of the second cardiac image is calculated.

6. The method as claimed in claim 4, wherein the MR signals generated at the echo time TE₁ and the MR signals generated at the echo time TE₂ are acquired such, that the phase of the nuclear spins of the fat protons and of the water protons is essentially the same during the acquisition of the corresponding MR signals.

7. The method as claimed in claim 1, the SPGR sequence comprising fat presaturation pulses which are applied in at least a part, preferably a large part, of the segments, in order to suppress the MRI signal of fat protons.

8. The method as claimed in claim 1, the RF pulses being applied such, that they selectively excite water protons leaving fat protons essentially unexcited.

9. The method as claimed in claim 1, wherein the MR signals are acquired asymmetrically in frequency encoding direction.

10. The method as claimed in claim 1, wherein the SPGR sequence is synchronized with the measured cardiac cycle indicator such, that the MR signals used for the reconstruction of the at least one first cardiac image are generated at end diastole.

11. The method as claimed in claim 1, the SPGR sequence comprising long-T₂ presaturation RF pulses which are applied in the form of long-T₂ suppression pulses, in order to suppress the MR signal of components with long transverse relaxation times (T₂).

12. The method as claimed in claim 1, wherein the MR signals are generated and acquired such, that the reconstructed first cardiac image reflects a rectangular field of view.

13. A magnetic resonance imaging (MRI) system at least comprising
a magnet for generating a main magnetic field at a location of a human or an animal heart to be imaged, in order to at least partly align nuclear spins of the heart;
an excitation module for applying radio frequency (RF) pulses to the heart, in order to excite the nuclear spins of the heart;
a gradient module for generating temporary magnetic gradient fields at a location of the heart;
an acquisition module for acquiring the magnetic resonance (MR) signals produced by excited nuclear spins of the sample;
a cardiac cycle measurement module for measuring at least one cardiac cycle indicator which reflects the timing of the cardiac cycles of the heart;
a control module configured for controlling the excitation module, the gradient module, the acquisition module and the cardiac cycle measurement module such, that the heart is subjected to a spoiled gradient echo (SPGR) sequence, the SPGR sequence being segmented into segments, wherein each segment is synchronized with the measured cardiac cycle indicator and comprises a plurality of basic sequence elements in each of which a radiofrequency (RF) pulse and a frequency encoding gradient moment kx are applied, in order to generate a magnetic resonance (MR) signal at an echo time TE₁ after the RF pulse, and wherein the RF pulses and the frequency encoding gradient moments kx are applied such, that in different basic sequence elements the MR signal is generated and acquired at varying echo times TE₁, in order to reduce the effective echo time in the center of k-space; and
a reconstruction module for reconstructing at least one image based on the acquired MR signals.

14. A computer program, for controlling a magnetic resonance imaging (MRI) system, in order to image components with short transverse relaxation times (T₂) in a human or an animal heart, the computer program at least comprising executable instructions to:
employ a spoiled gradient echo (SPGR) sequence on the MRI system, the SPGR sequence being segmented into segments, wherein each segment comprises a plurality of basic sequence elements in each of which a radiofrequency (RF) pulse and a frequency encoding gradient moment kx are applied, in order to generate a magnetic resonance (MR) signal at an echo time TE₁ after the RF pulse;
apply the RF pulses and the frequency encoding gradient moments kx such, that in different basic sequence elements the MR signal is generated at varying echo times TE₁, in order to reduce the effective echo time in the center of k-space;
measure at least one cardiac cycle indicator which reflects the timing of the cardiac cycles of the human or of the animal heart;
synchronize the segments of the SPGR sequence with the measured cardiac cycle indicator;
acquire the MR signals generated at the varying echo times TE₁; and
reconstruct at least one first cardiac image based on the acquired MR signals.
